(19) United States
(12) Patent Application Publication
(10) Pub. No.: US 2025/0273306 A1
(43) Pub. Date: Aug. 28, 2025

(54) ACCURATE PREDICTION OF GAS HYDRATE FORMATION CONDITIONS WITH ARTIFICIAL NEURAL NETWORKS (ANN) AND MULTILAYER PERCEPTRONS (MLPS)

(71) Applicant: Saudi Arabian Oil Company, Dhahran (SA)

(72) Inventors: James Ohioma Arukhe, Dhahran (SA); Bander Khonain, Dhahran (SA); Adnan Alsayedali, Qatif (SA)

(21) Appl. No.: 18/587,213

(22) Filed: Feb. 26, 2024

(51) Int. Cl. G16C 20/30; G16C 20/70

(52) U.S. Cl. CPC G16C 20/30; G16C 20/70

(57) ABSTRACT

The determination of the probability of gas hydrate formation using artificial neural network (ANN) and multilayer perceptrons (MLPs) models. ANN generally refers to a network of interconnected neurons (also referred to as “nodes”) that model the neurons in a human brain. An MLP refers to a feed-forward network having a specific arrangement of neurons and includes an input layer, one or more hidden layers, and an output layer. Input data such as temperature, pressure, gas mixture composition, and indicators of gas hydrate formation may be obtained and preprocessed for use in training and testing. The ANN and MLPs may be trained using a training set of the input to output a probability of gas hydrate formation. The trained ANN and MLP models may then be used to determine a gas hydrate formation probability for new data associated with a pipeline transporting a gas mixture.

### BACKGROUND

### Field of the Disclosure

The present disclosure generally relates to the production and transport of hydrocarbons such as oil and gas from hydrocarbon-bearing reservoirs. More specifically, embodiments of the disclosure relate to predicting the formation of gas hydrates in pipelines used to transport hydrocarbons.

### Description of the Related Art

A hydrocarbon reservoir is a pool of hydrocarbons (for example, oil or gas) trapped in a subsurface rock formation. Hydrocarbon wells are often drilled into hydrocarbon reservoirs to extract (or “produce”) the trapped hydrocarbons, which are then transported for processing and use. For example, production pipelines may be used to transport oil and gas and can extend for thousands of kilometers between reservoirs and oil and gas terminals. In some cases, the flow of oil and gas may become partially or completely blocked at certain locations along a production pipeline due to sedimentation of various substances along the pipeline, such as gas hydrates. Gas hydrates are crystalline solids formed from water and natural gas, most commonly methane, under certain pressure and temperature conditions. They are found in large quantities in nature, both in permafrost regions and under the sea floor. If gas hydrates form in pipelines, they can cause blockages that are difficult to remove and can potentially lead to significant equipment damage and eventually catastrophic failures.

### SUMMARY

Existing techniques for predicting gas hydrate formation conditions typically involve thermodynamic models. These models require extensive knowledge about the composition of the gas and water mixture, as well as the precise conditions of temperature and pressure. However, these models are complex and time-consuming to use. Moreover, these models often fail to accurately predict the conditions for hydrate formation in systems with high levels of impurities or in conditions far from equilibrium.

Artificial intelligence, such as artificial neural networks (ANN), may be used to improve prediction accuracy. However, these technologies face challenges due to lack of comprehensive and high-quality training data, as the conditions for gas hydrate formation can be difficult and expensive to measure accurately in a laboratory setting. The limited availability data can make it difficult to train an effective artificial intelligent model. Moreover, artificial neural network solutions fail to interpret and explain the resulting predictions, making it difficult for engineers to trust and use the predictions and the model.

In one embodiment, a method for determining the probability of gas hydrate formation in a pipeline is provided. The method includes obtaining a first plurality of parameters and respective values associated with a gas mixture, the first plurality of parameters including temperature, pressure, a composition of the gas mixture, and an indication of gas hydrate formation. The method also includes processing the plurality of parameters and respective values to obtain a training dataset and testing dataset and training a neural network using the plurality of parameters and respective values of the training dataset to create a gas hydrate formation model. The method further includes using the trained gas hydrate formation model to determine a gas hydrate formation probability for the pipeline from a second plurality of parameters and respective values associated with the pipeline.

In some embodiments, processing the plurality of parameters and respective values includes normalizing the plurality of respective values to a range. In some embodiments, the method includes using the trained gas hydrate formation model to determine a temperature of gas hydrate formation and a pressure of gas hydrate formation. In some embodiments, the neural network is a feed-forward neural network. In some embodiments, the neural network includes a multilayer perceptron. In some embodiments, the method includes evaluating the gas hydrate formation model before using the gas hydrate formation model to determine the gas hydrate formation probability for the pipeline, such that evaluating the gas hydrate formation model include calculating a metric between a gas hydrate formation probability produced by the gas hydrate formation model and an indication of gas hydrate formation in the training data. The metric may include a mean absolute error, a mean squared error, or a root mean squared error. In some embodiments, the method also includes adjusting a temperature or pressure associated with the pipeline based on the gas hydrate formation probability.

In another embodiment, a non-transitory computer readable storage medium having program instructions stored thereon for determining the probability of gas hydrate formation in a pipeline is provided. The program instructions are executable by a processor to perform operations that include obtaining a first plurality of parameters and respective values associated with a gas mixture, the first plurality of parameters including temperature, pressure, a composition of the gas mixture, and an indication of gas hydrate formation. The operations also include processing the plurality of parameters and respective values to obtain a training dataset and testing dataset and training a neural network using the plurality of parameters and respective values of the training dataset to create a gas hydrate formation model. The operations further include using the trained gas hydrate formation model to determine a gas hydrate formation probability for the pipeline from a second plurality of parameters and respective values associated with the pipeline.

In some embodiments, processing the plurality of parameters and respective values includes normalizing the plurality of respective values to a range. In some embodiments, the operations include using the trained gas hydrate formation model to determine a temperature of gas hydrate formation and a pressure of gas hydrate formation. In some embodiments, the neural network is a feed-forward neural network. In some embodiments, the neural network includes a multilayer perceptron. In some embodiments, the operations include evaluating the gas hydrate formation model before using the gas hydrate formation model to determine the gas hydrate formation probability for the pipeline, such that evaluating the gas hydrate formation model include calculating a metric between a gas hydrate formation probability produced by the gas hydrate formation model and an indication of gas hydrate formation in the training data. The metric may include a mean absolute error, a mean squared error, or a root mean squared error. In some embodiments, the operations include adjusting a temperature or pressure associated with the pipeline based on the gas hydrate formation probability.

In another embodiment, a system for determining the probability of gas hydrate formation in a pipeline is provided. The system includes a processor and a non-transitory computer-readable memory accessible by the processor and having executable code stored thereon. The executable code includes a set of instructions that causes the processor to perform operations that include obtaining a first plurality of parameters and respective values associated with a gas mixture, the first plurality of parameters including temperature, pressure, a composition of the gas mixture, and an indication of gas hydrate formation. The operations also include processing the plurality of parameters and respective values to obtain a training dataset and testing dataset and training a neural network using the plurality of parameters and respective values of the training dataset to create a gas hydrate formation model. The operations further include using the trained gas hydrate formation model to determine a gas hydrate formation probability for the pipeline from a second plurality of parameters and respective values associated with the pipeline.

In some embodiments, processing the plurality of parameters and respective values includes normalizing the plurality of respective values to a range. In some embodiments, the operations include using the trained gas hydrate formation model to determine a temperature of gas hydrate formation and a pressure of gas hydrate formation. In some embodiments, the neural network is a feed-forward neural network. In some embodiments, the neural network includes a multilayer perceptron. In some embodiments, the operations include evaluating the gas hydrate formation model before using the gas hydrate formation model to determine the gas hydrate formation probability for the pipeline, such that evaluating the gas hydrate formation model include calculating a metric between a gas hydrate formation probability produced by the gas hydrate formation model and an indication of gas hydrate formation in the training data. The metric may include a mean absolute error, a mean squared error, or a root mean squared error. In some embodiments, the operations include adjusting a temperature or pressure associated with the pipeline based on the gas hydrate formation probability.

### BRIEF DESCRIPTION OF THE DRAWINGS

FIG. 1 is a schematic diagram of a gas pipeline and gas hydrate formation model in accordance with an embodiment of the disclosure;

FIG. 2 is a flowchart of a process using artificial neural network (ANN) and multilayer perceptrons (MLPs) gas hydrate models in accordance with an embodiment of the disclosure;

FIG. 3 is a flowchart of a process for generating and using artificial neural network (ANN) and multilayer perceptrons (MLPs) gas hydrate models to determine gas hydrate formation in accordance with an embodiment of the disclosure;

FIG. 4 is a block diagram of a data processing system in accordance with an embodiment of the disclosure;

FIG. 5 is a bar graph comparing the performance metrics and values for an ANN model and an MLP model in accordance with an embodiment of the disclosure; and

FIG. 6 is a graph comparing the prediction accuracy for an ANN model and an MLP model over time in accordance with an embodiment of the disclosure.

### DETAILED DESCRIPTION

The present disclosure will be described more fully with reference to the accompanying drawings, which illustrate embodiments of the disclosure. This disclosure may, however, be embodied in many different forms and should not be construed as limited to the illustrated embodiments. Rather, these embodiments are provided so that this disclosure will be thorough and complete, and will fully convey the scope of the disclosure to those skilled in the art.

Embodiments of the disclosure are directed to the prediction (that is, determination) of the likelihood (that is, probability) of the formation of gas hydrates in a pipeline using an artificial neural network (ANN) model and multilayer perceptrons (MLPs) model. The probability of gas hydrate formation may be expressed as a percentage or a probability score between 0 and 1, such that a greater value indicates a greater probability of gas hydrate formation based on input parameters provided to the models. In some embodiments, the model may output a formation temperature, a formation pressure, a formation time, or any combination thereof, in combination with the gas hydrate formation probability.

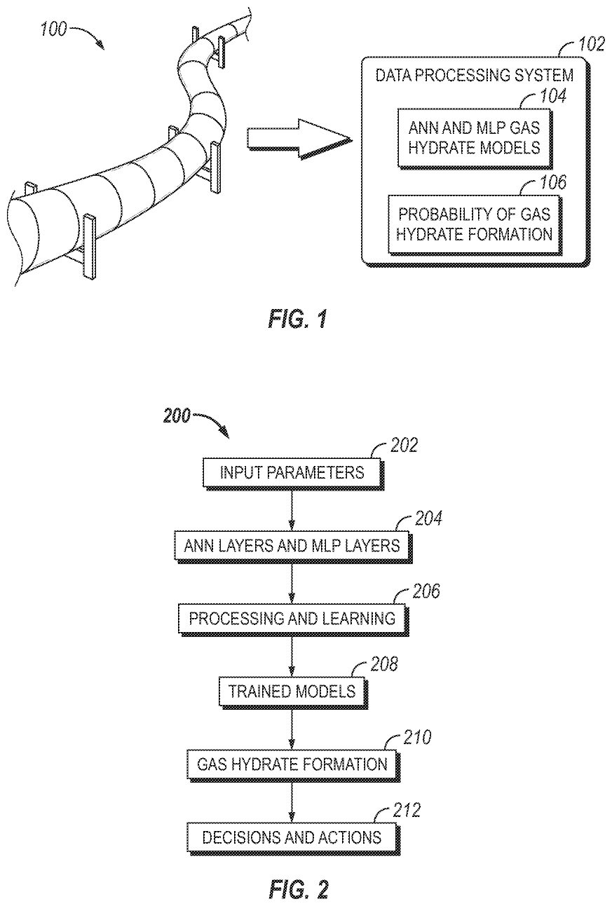

FIG. 1 is a diagram that illustrates a gas pipeline 100 in accordance with an embodiment of the disclosure. In the illustrated embodiment, the gas pipeline 100 may be representative of a gas pipeline network that transports natural gas from one location (for example, a production site) to another location (for example, distribution sites or industrial facilities). The gas pipeline 100 may transport natural gas that may include other components such as water, resulting in the susceptibility of the pipeline 100 to gas hydrate formation.

FIG. 1 also depicts a data processing system 102 that may be operable to receive various parameters associated with the pipeline or the gas mixture in the pipeline, such as temperature, pressure, and gas mixture composition, and make various determinations. For example, the pipeline 100 may include temperature sensors, pressure sensors, and fluid sampling capabilities to provide temperature, pressure, and gas mixture composition to systems such as the data processing system 102.

The data processing system 102 may include memory and a processor that capable of performing the various processes described in the disclosure. Accordingly, the data processing system 102 may include an artificial neural network (ANN) and multilayer perceptrons (MLPs) gas hydrate models 104 implemented according to the techniques of the disclosure. As described in the disclosure, ANN and MLPs gas hydrate models may be used to determine the probability of gas hydrate formation (106) in the pipeline 100.

FIG. 2 depicts a process 200 for determining gas hydrate formation using ANN and MLPs gas hydrate models in accordance with an embodiment of the disclosure. Initially, input parameters may be obtained (block 202). The input parameters may include the composition of the mixture in a pipeline, the temperature, the pressure, and indicators of gas hydrate formation. In some embodiments, the input parameters are determined from laboratory measurements, field observations or both. For example, gas hydrate formation for a known composition under controlled temperature and pressure may be tested in a laboratory by varying the temperature, pressure, composition, or any combination thereof and observing whether gas hydrates form.

The input parameters may then be provided to ANN and MLPs layers (block 204). As discussed in the disclosure, the models may include a number of layers for receiving and processing the inputs. After processing (for example, standardizing) the input parameters, weights and biases may be assigned to the normalized input parameters. For example, essential relationships may be constructed linearly using Equations 1 and 2 as follows:

$$Normalized Hydrate {Temp}_{\cdot n} = {\sum }_{i = 1}^{n} {W}_{2 i} ( {W}_{1 i 1} {P}_{n} + {W}_{1 i 2} {GG}_{n} + {b}_{1 , i} ) + {b}_{2} \qquad ( 1 )$$

$$Normalized Hydrate {Press}_{\cdot n} = {\sum }_{i = 1}^{n} {W}_{2 i} ( {W}_{1 i 1} {T}_{n} + {W}_{1 i 2} {GG}_{n} + {b}_{1 , i} ) + {b}_{2} \qquad ( 2 )$$

Where W is the weights created by the neural network, b is the biases created by the neural network, n is the count of neurons in the first hidden layer, i is the index of each neuron in the hidden layer, Tₙ is the standardized temperature, Pₙ is the standardized pressure, and GGₙ is the gas gravity. For example, Wii is the weight related to each input parameter for the input and hidden layers, b₁ᵢ is the bias associated with the input and training layers, W₂ᵢ is the weight associated with the hidden and output layers, and b₂ᵢ is the bias associated with the hidden and output layers.

The models may process and learn from the input parameters (block 206) to produce trained models (block 208). The training of the models allows the models to recognize patterns and make predictions based on the input data. In some embodiments, the training may include adjusting weights and biases of the connections in the layers of the ANN and MLPs gas hydrate models based on the error in the model's predictions.

After processing and learning, the trained models may be used to predict the likelihood of gas hydrate formation in a pipeline when provided a new set of input parameters from a pipeline (block 210). After processing the new set of input parameters, the output from the models is the probability of gas hydrate formation under the conditions of the new input parameters. The prediction may be used to take action or make decisions regarding the potential gas hydrate formations (block 212). For example, based on the probability of gas hydrate formation, operating conditions may be adjusted or preventative measures taken to avoid gas hydrate formation in the pipeline. For example, the temperature of the pipeline may be actively managing, such as by insulating the pipeline, heating the pipeline or both. In some instances, a hydrate inhibitor (including low dosage hydrate inhibitors (LDHIs)) may be added to the gas mixture in the pipeline. Such hydrate inhibitors may include methanol, ethanol, glycols, and salts. In another instance, the pressure in the pipeline may be maintained, such as by increasing or decreasing the pressure based on considerations of the temperature (for example, increasing the pressure may raise the hydrate formation temperature).

In some embodiments, the ANN and MLPs gas hydrate models may be used in pipeline management. For example, the models may be used to prevent blockages and maintain the efficiency and safety of operations in pipeline operations. In some embodiments, the ANN and MLPs gas hydrate models may also be used in exploration for and production of hydrocarbon resources such as gas. In such instances, the determination of gas hydrate formation may be used to implement preventative measures and reduce the risks associated with the formation. In some embodiments, the ANN and MLPs gas hydrate models may be used in research and development environments to predict the formation of has hydrates in these settings for use as an energy source or to determine their impact on climate change.

The primary output of the ANN and MLPs gas hydrate model is the likelihood (that is, probability) of gas hydrate formation. In some embodiments, the probability of gas hydrate formation may be expressed as a percentage or a probability score between 0 and 1, such that a greater value indicates a greater probability of gas hydrate formation.

In some embodiments, the ANN and MLPs gas hydrate models may provide other outputs that provide additional information about gas hydrate formation. In some embodiments, the ANN and MLPs gas hydrate models may provide an output of formation temperature—the specific temperature at which gas hydrates are likely to form under the given conditions. In some embodiments, the ANN and MLPs gas hydrate models may provide an output of formation pressure—the specific pressure at which gas hydrates are likely to form under the given conditions. In some embodiments, the ANN and MLPs gas hydrate models may provide an output of formation time—the amount of time for which gas hydrates are likely to form under the given conditions.

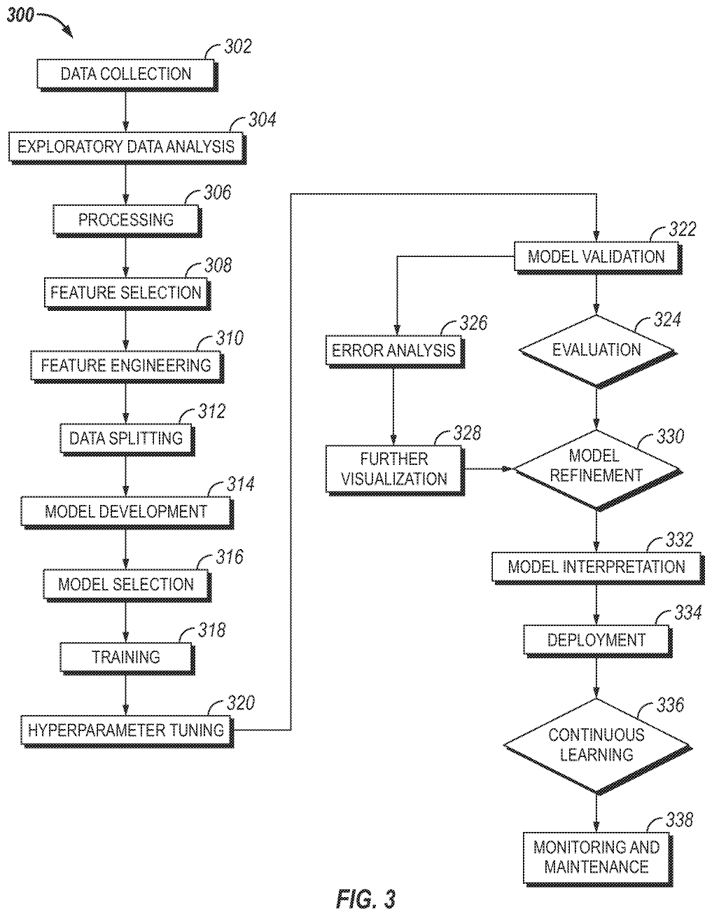

FIG. 3 depicts a process 300 for generating and using ANN and MLPs gas hydrate models in accordance with an embodiment of the disclosure. Initially, data relevant to gas hydrate formation is collected (block 302). The data may include temperature, pressure, and gas-water mixture composition. In some embodiments, the data may be collected from lab measurements, field observations, or a combination thereof. In some embodiments, the collected data may include collection from sources having large levels of impurities and non-equilibrium conditions.

Next the data is analyzed (block 304) to determine the structure, relationships between variables, potential outliers, and anomalies. The data analysis may include statistical analysis and generating statistical summaries and visualizations.

As shown in FIG. 3, the data may be preprocessed (block 306) to prepare the data for the models. This may include cleaning the data by processing missing values and outliers in the data, and normalizing numerical data. In some embodiments, processing missing data may include filling in missing values using an average value for a variable or using interpolation to estimate the missing values. In some embodiments, outliers may be deleted from the data, replaced with a median value, or replaced with a value based on domain knowledge or expert opinion. In some embodiments, data normalization may include transforming all values of the input data to a common scale. In some embodiments, the common scale is 0 to 1.

Next, the most relevant features for the ANN and MLPs gas hydrate models are selected (block 308). In some embodiments, this may be done based on the correlation of the features with the target variable (that is, probability of gas hydrate formation) or using other feature selection techniques. Additionally, the process 300 may include feature engineering (block 310). The feature engineering may include creating new features from the existing data that might improve the performance of the machine learning model. In some embodiments, the feature engineering may include combining features, creating polynomial features, creating dummy variables for categorical features, or any combination thereof.

Next, the preprocessed data may be split into a training set and testing set (block 312). In some embodiments, 80% of the data may be used for training the model and 20% of the data may be used for testing the model's performance. In some embodiments, 70% of the data may be used for training the model and 30% of the data may be used for testing the model's performance. Other embodiments may use different ratios of training data to testing data. As discussed in the disclosure, the training data may be used in the process 300 for training the models, while the testing data is used to test the trained models.

The ANN and MLPs gas hydrate models may then be developed using the training data (block 314). Both the ANN model and MLP model may use the temperature and pressure weighting functions described supra in Equations 1 and 2. In some embodiments, the artificial neural network (ANN) model refers to a general ANN model that may have a single layer perceptron having a single layer of nodes connected to the inputs and that produces an output. Each node may be connected to an input and associated with the weights, such as the weights described in the disclosure for normalized temperature and normalized pressure. The output of each neuron (also referred to as “node”) may be determining by computing the weighted sum and the inputs, and the output of each neuron may be provided to an activation function to introduce non-linearity in the model. In some embodiments, the activation function may be a step function or sigmoid function. The activation function may then provide the output (that is, gas hydrate formation probability).

The MLP model is a feed-forward network and may include an input layer, one or more hidden layers, and an output layer. In some embodiments, the input layer may include a number of neurons corresponding to the number of input variables in the input layer. For example, for embodiments having three input variables, three neurons may be used in the input layer. The neurons in the hidden layers may use activation functions to introduce non-linearity into the model. In some embodiments, the activation functions may include ReLU (Rectified Linear Unit) or tan h (hyperbolic tangent). The output layer may include one neuron corresponding to the output variable (gas hydrate formation probability). The output layer may include an activation function corresponding to the nature of the problem (for example, regression or classification). In some embodiments, the activation function for a regression problem may be a linear activation function or an identity activation function.

Next, as shown in FIG. 3, the artificial neural network (ANN) model, multilayer perceptrons (MLPs) model, or both are selected (block 316). The training data may then be used to train a model (block 318). In some embodiments, the hyperparameters of the model may be tuned (block 320) during training to improve the performance of the model. In some embodiments, tuning the hyperparameters may include grid search, random search, or a combination thereof. The tuning may include using a suitable learning algorithm (to adjust the weights and biases of the model during training. In some embodiments, the learning algorithm is backpropagation.

Next, the trained ANN and MLPs gas hydrate models may be validated (block 322). In some embodiments, the validation may include cross-validation by splitting the data into several subsets for training and testing and then training and testing the model on different combinations of these subsets. The validated models may then be evaluated (block 324) based on its performance on the testing data. Based on the evaluation, the process 300 may proceed to the model refinement (block 330).

In some embodiments, error metrics may be determined (block 326) to assess the accuracy of the determinations from the model. The metrics may include accuracy, precision, recall, F1-score, or other suitable metrics or combination thereof. In some embodiments, the metrics used to evaluate the determination of gas hydrate formation probability may include Mean Absolute Error (MAE), Mean Squared Error (MSE), or Root Mean Squared Error (RMSE). The metrics may be determined by comparing a model's gas hydrate formation probability and the actual gas hydrate formation probability in the testing dataset, such that lower values for the metrics would indicate better model performance. In some embodiments, visualizations may be generated (block 328) to analyze and interpret the results from the model. The visualizations may include visualizations of the training process, performance plots, regression plots, cross plots, or any combination thereof.

As shown in FIG. 3, the process 300 may include refinement of the models (block 330). The model refinement includes tuning the models based on the evaluation results. The model refinement may include adjusting a model's hyperparameters, adding or removing features from a model, or adjusting the architecture of the ANN or MLP of a respective model. For example, to address underfitting the architecture of the ANN or MLP, may be adjusted, such as by increasing complexity by adding hidden layers, neurons in each layer, or both. In another example, to address overfitting, the hyperparameters may be tuned to reduce the learning rate. Hyperparameters that may be adjusted may include the batch size, number of training iterations, or the type of optimizer used in the training phase.

Next, the models' predictions may be interpreted (block 332). This may include analyzing the importance of different features in the models' predictions. As shown in FIG. 3, the ANN and MLPs gas hydrate models may then be deployed (block 334). As discussed in the disclosure, the models may output a probability of gas hydrate formation for a new input data. In some embodiments, the models may output a formation temperature, a formation pressure, a formation time, or any combination thereof, to assist with managing gas hydrate formation in certain applications.

In some embodiments, the process 300 includes continuous learning (block 336) and monitoring and maintenance (block 338), such that the models continually learn and improve to account for new data, changes pipeline or gas conditions, and other factors. This may include retraining the neural network using additional data. The additional data may be processed in the same manner as the initial data so that the network adjusts its weights and biases to better fit the new data.

Advantageously, the trained ANN and MLPs gas hydrate models may provide improved accuracy of predictions of gas hydrate formations as compared to the existing techniques, especially in systems having more complex conditions. Moreover, by training the models on data from gas hydrate systems that have large levels of impurities and non-equilibrium conditions, the trained ANN and MLPs gas hydrate models may be able to accurately process a wide variety of scenarios with improved accuracy. The trained ANN and MLPs gas hydrate models are also adaptable to new data or changing conditions, in contrast to existing static models. Moreover, the trained ANN and MLPs gas hydrate models may process input data and provide probability determinations significantly faster and more efficiently than traditional thermodynamic models, which are more complex and time-consuming to use.

Additionally, in some implementations, the use of the trained ANN and MLPs gas hydrate models may help mitigate the risks of gas hydrate formation, including environmental risks, and improve operational efficiency. Further, by reducing the occurrences of gas hydrate formation and the resulting blockages, equipment damage, and operational disruptions before they occur, the trained ANN and MLPs gas hydrate models may reduce costs for the oil and gas industry as compared to the existing technologies for addressing gas hydrate formation. The trained ANN and MLPs gas hydrate models may also be sufficiently scalable in terms of amounts of data so that they are suitable use in large-scale industrial operations.

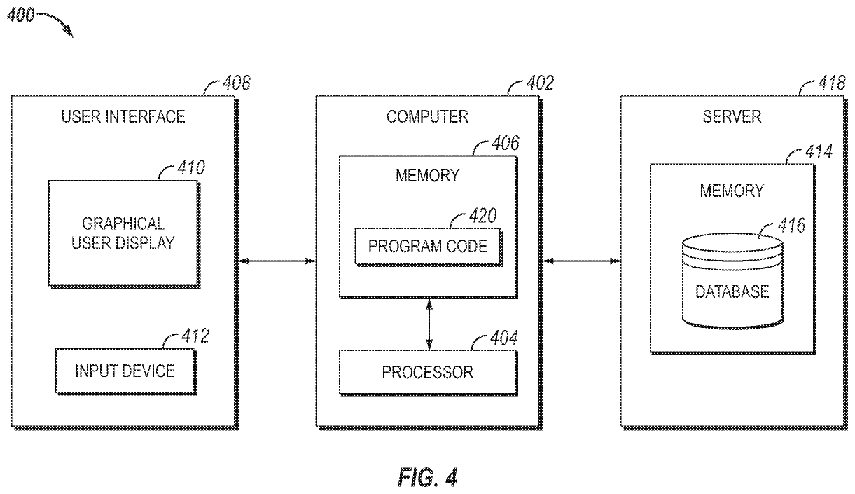

FIG. 4 depicts a data processing system 400 (such as the data processing system 102) that includes a computer 402 having a processor 404 and memory 406 coupled to the processor 404 to store operating instructions, control information and database records therein in accordance with an embodiment of the disclosure. The data processing system 400 may be a multicore processor with nodes such as those from Intel Corporation or Advanced Micro Devices (AMD), or an HPC Linux cluster computer. The data processing system 400 may also be a mainframe computer of any conventional type of suitable processing capacity such as those available from International Business Machines (IBM) of Armonk, N.Y., or other source. The data processing system 400 may in cases also be a computer of any conventional type of suitable processing capacity, such as a personal computer, laptop computer, or any other suitable processing apparatus. The data processing system 400 may also be representative of resources available in a computer cluster or a cloud-computing platform. It should thus be understood that a number of commercially available data processing systems and types of computers may be used for this purpose.

The computer 402 is accessible to operators or users through user interface 408 and are available for displaying output data or records of processing results obtained according to the present disclosure with an output graphic user display 410. The output display 410 includes components such as a printer and an output display screen capable of providing printed output information or visible displays in the form of graphs, data sheets, graphical images, data plots and the like as output records or images.

The user interface 408 of computer 402 also includes a suitable user input device or input/output control unit 412 to provide a user access to control or access information and database records and operate the computer 402. Data processing system 400 further includes a database of data stored in computer memory, which may be internal memory 406, or an external, networked, or non-networked memory as indicated at 414 in an associated database 416 in a server 418.

The data processing system 400 includes executable code 420 stored in non-transitory memory 406 of the computer 402. The executable code 420 according to the present disclosure is in the form of computer operable instructions causing the data processor 404 to receive input data and provide outputs based on processing the input data. The computer operable instructions of the executable code 420 may execute and train ANN and MLPs gas hydrate models according to the techniques described in the disclosure, and may determine a gas hydrate formation probability using the ANN and MLP gas hydrate models.

### EXAMPLES

The following examples are included to demonstrate embodiments of the disclosure. It should be appreciated by those of skill in the art that the techniques and compositions disclosed in the example which follows represents techniques and compositions discovered to function well in the practice of the disclosure, and thus can be considered to constitute modes for its practice. However, those of skill in the art should, in light of the present disclosure, appreciate that many changes can be made in the specific embodiments which are disclosed and still obtain a like or a similar result without departing from the spirit and scope of the disclosure.

Table 1 depicts an example of data collected from laboratory experiments that may be used as an input data in embodiments of the disclosure:

TABLE 1
EXAMPLE INPUT DATA

| | | | Gas | Hydrate |
|---|---|---|---|---|
| Experiment | Temperature | Pressure | Composition | Formed |
| No. | (° C.) | (MPa) | (%) | (Yes/No) |
| 1 | −2 | 7.5 | CH4: 70, CO2: 30 | Yes |
| 2 | 0 | 5 | CH4: 80, CO2: 20 | No |
| 3 | −3 | 6.5 | CH4: 75, CO2: 25 | Yes |
| 4 | −1 | 7 | CH4: 60, CO2: 40 | No |
| 5 | −2 | 6 | CH4: 70, CO2: 30 | Yes |
| 6 | 1 | 5.5 | CH4: 80, CO2: 20 | No |
| 7 | −4 | 7 | CH4: 75, CO2: 25 | Yes |
| 8 | −2 | 6.5 | CH4: 70, CO2: 30 | Yes |
| 9 | 0 | 5 | CH4: 80, CO2: 20 | No |
| 10 | −3 | 7.5 | CH4: 70, CO2: 30 | Yes |

As shown in Table 1, data may be collected from 10 experiments. The data includes temperature in degrees Celsius (° C.), pressure in megaPascals (MPa), and gas composition in percentages of CH₄ and CO₂.

Using the example data of Table 1, an artificial neural network (ANN) model and a multilayer perceptrons (MLPs) model trained according to the techniques of the disclosure may be evaluated and compared. The performance metrics and comparisons are shown in Table 2:

TABLE 2
MODEL PERFORMANCE METRICS

| | | MLP |
|---|---|---|
| Metric | ANN Model | Model |
| MSE | 0.03 | 0.05 |
| MAPE | 2% | 4% |
| RMSE | 0.15 | 0.2 |
| R2 | 0.95 | 0.9 |

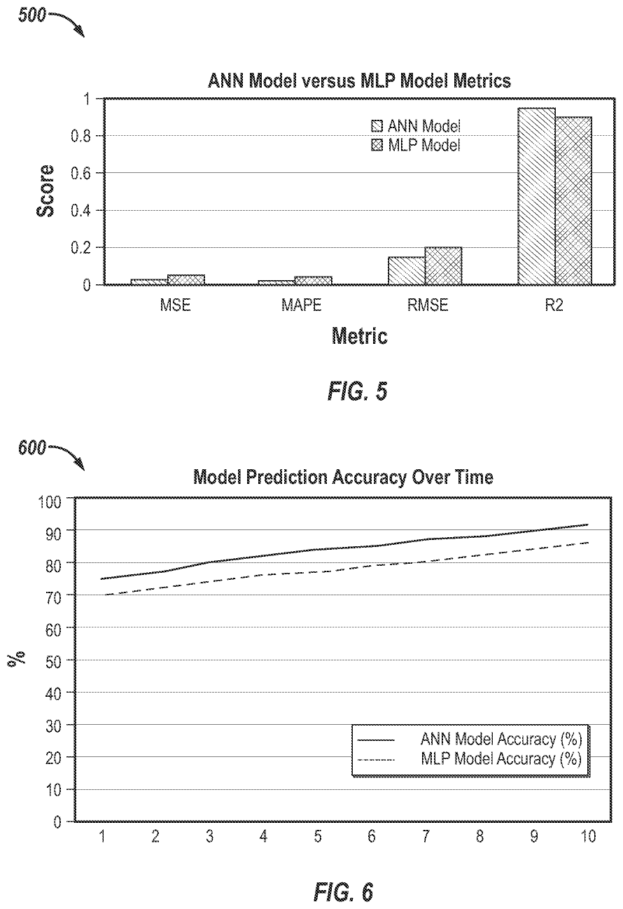

By way of example, FIG. 5 is a bar graph comparing the performance metrics and values of Table 2 for the ANN model and the MLP model.

Additionally, in the example, the prediction accuracy of the artificial neural network (ANN) model and a multilayer perceptrons (MLPs) model may be compared over time. The prediction accuracy over time (that is, for 10 trials) is shown in Table 3:

TABLE 3
MODEL PREDICTION ACCURACY OVER TIME

| Trial | ANN Model | MLP Model |
|---|---|---|
| No. | Accuracy (%) | Accuracy (%) |
| 1 | 75 | 70 |
| 2 | 77 | 72 |
| 3 | 80 | 74 |
| 4 | 82 | 76 |
| 5 | 84 | 77 |
| 6 | 85 | 79 |
| 7 | 87 | 80 |
| 8 | 88 | 82 |
| 9 | 90 | 84 |
| 10 | 92 | 86 |

By way of example, FIG. 6 is a graph 600 comparing the prediction accuracy for the ANN model and the MLP model over time.

Ranges may be expressed in the disclosure as from about one particular value, to about another particular value, or both. When such a range is expressed, it is to be understood that another embodiment is from the one particular value, to the other particular value, or both, along with all combinations within said range.

Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the embodiments described in the disclosure. It is to be understood that the forms shown and described in the disclosure are to be taken as examples of embodiments. Elements and materials may be substituted for those illustrated and described in the disclosure, parts and processes may be reversed or omitted, and certain features may be utilized independently, all as would be apparent to one skilled in the art after having the benefit of this description. Changes may be made in the elements described in the disclosure without departing from the spirit and scope of the disclosure as described in the following claims. Headings used in the disclosure are for organizational purposes only and are not meant to be used to limit the scope of the description.

What is claimed is:

1. A method for determining the probability of gas hydrate formation in a pipeline, comprising:
obtaining a first plurality of parameters and respective values associated with a gas mixture, the first plurality of parameters comprising temperature, pressure, a composition of the gas mixture, and an indication of gas hydrate formation;
processing the plurality of parameters and respective values to obtain a training dataset and testing dataset;
training a neural network using the plurality of parameters and respective values of the training dataset to create a gas hydrate formation model; and
using the trained gas hydrate formation model to determine a gas hydrate formation probability for the pipeline from a second plurality of parameters and respective values associated with the pipeline.

2. The method of claim 1, wherein processing the plurality of parameters and respective values comprising normalizing the plurality of respective values to a range.

3. The method of claim 1, comprising using the trained gas hydrate formation model to determine a temperature of gas hydrate formation and a pressure of gas hydrate formation.

4. The method of claim 1, wherein the neural network comprises a feed-forward neural network.

5. The method of claim 1, wherein the neural network comprises a multilayer perceptron.

6. The method of claim 1, comprising evaluating the gas hydrate formation model before using the gas hydrate formation model to determine the gas hydrate formation probability for the pipeline, wherein evaluating the gas hydrate formation model comprises:
calculating a metric between a gas hydrate formation probability produced by the gas hydrate formation model and an indication of gas hydrate formation in the training data, wherein the metric comprises a mean absolute error, a mean squared error, or a root mean squared error.

7. The method of claim 1, comprising:
adjusting a temperature or pressure associated with the pipeline based on the gas hydrate formation probability.

8. A non-transitory computer readable storage medium comprising program instructions stored thereon for determining the probability of gas hydrate formation in a pipeline, the program instructions executable by a processor to perform operations comprising:
obtaining a first plurality of parameters and respective values associated with a gas mixture, the first plurality of parameters comprising temperature, pressure, a composition of the gas mixture, and an indication of gas hydrate formation;
processing the plurality of parameters and respective values to obtain a training dataset and testing dataset;
training a neural network using the plurality of parameters and respective values of the training dataset to create a gas hydrate formation model; and
using the trained gas hydrate formation model to determine a gas hydrate formation probability for the pipeline from a second plurality of parameters and respective values associated with the pipeline.

9. The non-transitory computer readable storage medium of claim 8, wherein processing the plurality of parameters and respective values comprising normalizing the plurality of respective values to a range.

10. The non-transitory computer readable storage medium of claim 8, the operations comprising using the trained gas hydrate formation model to determine a temperature of gas hydrate formation and a pressure of gas hydrate formation.

11. The non-transitory computer readable storage medium of claim 8, wherein the neural network comprises a feed-forward neural network.

12. The non-transitory computer readable storage medium of claim 8, wherein the neural network comprises a multilayer perceptron.

13. The non-transitory computer readable storage medium of claim 8, the operations comprising evaluating the gas hydrate formation model before using the gas hydrate formation model to determine the gas hydrate formation probability for the pipeline, wherein evaluating the gas hydrate formation model comprises:
calculating a metric between a gas hydrate formation probability produced by the gas hydrate formation model and an indication of gas hydrate formation in the training data, wherein the metric comprises a mean absolute error, a mean squared error, or a root mean squared error.

14. The non-transitory computer readable storage medium of claim 8, the operations comprising:
adjusting a temperature or pressure associated with the pipeline based on the gas hydrate formation probability.

15. A system for determining the probability of gas hydrate formation in a pipeline, comprising:
a processor;
a non-transitory computer-readable memory accessible by the processor and having executable code stored thereon, the executable code comprising a set of instructions that causes the processor to perform operations comprising:
obtaining a first plurality of parameters and respective values associated with a gas mixture, the first plurality of parameters comprising temperature, pressure, a composition of the gas mixture, and an indication of gas hydrate formation;
processing the plurality of parameters and respective values to obtain a training dataset and testing dataset;
training a neural network using the plurality of parameters and respective values of the training dataset to create a gas hydrate formation model; and
using the trained gas hydrate formation model to determine a gas hydrate formation probability for the pipeline from a second plurality of parameters and respective values associated with the pipeline.

16. The system of claim 15, wherein processing the plurality of parameters and respective values comprising normalizing the plurality of respective values to a range.

17. The system of claim 15, the operations comprising using the trained gas hydrate formation model to determine a temperature of gas hydrate formation and a pressure of gas hydrate formation.

18. The system of claim 15, wherein the neural network comprises a feed-forward neural network.

19. The system of claim 15, wherein the neural network comprises a multilayer perceptron.

20. The system of claim 15, the operations comprising evaluating the gas hydrate formation model before using the gas hydrate formation model to determine the gas hydrate formation probability for the pipeline, wherein evaluating the gas hydrate formation model comprises:
calculating a metric between a gas hydrate formation probability produced by the gas hydrate formation model and an indication of gas hydrate formation in the training data, wherein the metric comprises a mean absolute error, a mean squared error, or a root mean squared error.

21. The system of claim 15, the operations comprising:
adjusting a temperature or pressure associated with the pipeline based on the gas hydrate formation probability.
